(19) United States
(12) Patent Application Publication
(10) Pub. No.: US 2024/0139005 A1
(43) Pub. Date: May 2, 2024

(54) ALIGNMENT TOOL FOR ALIGNING HEART VALVE WITH DELIVERY SYSTEM

(71) Applicant: BOSTON SCIENTIFIC SCIMED, INC., Maple Grove, MN (US)

(72) Inventors: Tim O'Connor, Galway (IE); Enda Hannon, Galway (IE); John Lardner, Galway (IE); Declan Loughnane, Galway (IE); Pearse A. Coffey, Galway (IE)

(73) Assignee: BOSTON SCIENTIFIC SCIMED, INC., MAPLE GROVE, MN (US)

(21) Appl. No.: 18/493,299

(22) Filed: Oct. 24, 2023

Related U.S. Application Data
(60) Provisional application No. 63/419,400, filed on Oct. 26, 2022.

(51) Int. Cl. A61F 2/95; A61F 2/24

(52) U.S. Cl. CPC A61F 2/9522; A61F 2/2418; A61F 2/2427; A61F 2002/9505

(57) ABSTRACT

An alignment tool includes a body and a plurality of securement arms extending from the body, each of the plurality of securement arms adapted to releasably secure the alignment tool relative to a loader. The alignment tool includes a plurality of alignment arms extending from the body, each of the plurality of alignment arms including a first end having an alignment slot and an opposing second end including a handle portion, the plurality of alignment arms movable between a closed configuration defining a minimum distance between the first end of each of the plurality of alignment arms and an open configuration defining a maximum distance between the first end of each of the plurality of alignment arms. The plurality of alignment arms are biased into the closed configuration.

### CROSS REFERENCE TO RELATED APPLICATIONS

This application claims the benefit of priority of U.S. Provisional Application No. 63/419,400 filed Oct. 26, 2022, the entire disclosure of which is hereby incorporated by reference.

### TECHNICAL FIELD

The disclosure pertains to medical devices and more particularly to devices for aligning a heart valve during loading into a delivery system, and methods for using such medical devices.

### BACKGROUND

A wide variety of medical devices have been developed for medical use including, for example, artificial heart valves for repair or replacement of diseased heart valves. The artificial heart valve must be aligned precisely as it is loaded into a delivery system. Of the known medical devices and methods, each has certain advantages and disadvantages. There is an ongoing need to provide alternative medical devices as well as alternative methods for manufacturing and using the medical devices.

### SUMMARY

This disclosure provides design, material, manufacturing method, and use alternatives for medical devices. An example may be found in an alignment tool for loading a stent having a plurality of alignment loops onto a delivery catheter having a corresponding plurality of alignment pins adapted to be received within a corresponding alignment loop, the stent disposed within a loader having a loader housing. The alignment tool includes a body and a plurality of securement arms extending from the body, each of the plurality of securement arms adapted to releasably secure the alignment tool relative to the loader. A plurality of alignment arms extend from the body, each of the plurality of alignment arms including a first end having an alignment slot and an opposing second end including a handle portion, the plurality of alignment arms movable between a closed configuration defining a minimum distance between the first end of each of the plurality of alignment arms and an open configuration defining a maximum distance between the first end of each of the plurality of alignment arms. The plurality of alignment arms are biased into the closed configuration.

Alternatively or additionally, the alignment tool may further include a biasing member that biases the plurality of alignment arms into the closed configuration.

Alternatively or additionally, the biasing member may include a garter spring extending around each of the plurality of alignment arms.

Alternatively or additionally, the biasing member may include an elastomeric member extending around each of the plurality of alignment arms.

Alternatively or additionally, the biasing member may include a separate biasing member secured relative to each of the plurality of alignment arms.

Alternatively or additionally, the loader housing may include a face plate with which the plurality of securement arms are adapted to engage with when securing the alignment tool to the loader, the face plate adapted to permit the alignment tool to be secured relative to the loader within a range of relative rotational positions.

Alternatively or additionally, the plurality of securement arms may be monolithically formed as part of the body.

Alternatively or additionally, the plurality of alignment arms may be pivotably secured relative to the body.

Another example may be found in an alignment tool for loading a replacement cardiac valve having a plurality of alignment loops onto a delivery catheter having a corresponding plurality of alignment pins adapted to be received within a corresponding alignment loop, the replacement cardiac valve disposed within a loader having a loader housing. The alignment tool includes a body that is adapted to be releasably securable to the loader. A plurality of alignment arms extend from the body, each of the plurality of alignment arms including a first end having an alignment slot and an opposing second end including a handle portion, the plurality of alignment arms movable between a closed configuration defining a minimum distance between the first end of each of the plurality of alignment arms and an open configuration defining a maximum distance between the first end of each of the plurality of alignment arms. A biasing member is adapted to bias the plurality of alignment arms into the closed configuration.

Alternatively or additionally, the body may include one or more securement arms that are adapted to releasably secure the alignment tool relative to the loader.

Alternatively or additionally, the body may include an annular structure that is adapted to releasably secure the alignment tool relative to the loader.

Alternatively or additionally, the biasing member may include an annular spring.

Alternatively or additionally, the biasing member may include an O-ring.

Alternatively or additionally, the plurality of alignment arms may be adapted to move from the closed configuration to the open configuration in response to an inward force applied to the second ends of each of the plurality of alignment arms.

Alternatively or additionally, the second ends of each of the plurality of alignment arms may be adapted to accommodate being squeezed together in order to move from the closed configuration to the open configuration.

Alternatively or additionally, the plurality of alignment arms may be adapted to pivot between the closed configuration and the open configuration.

Alternatively or additionally, the alignment tool may further include a plurality of securement arms extending from the body, each of the plurality of securement arms adapted to releasably secure the alignment tool relative to the loader.

Alternatively or additionally, the body and the plurality of securement arms may be molded together.

Alternatively or additionally, the plurality of securement arms may be adapted to engage with a face plate of the loader when securing the alignment tool to the loader, the face plate adapted to permit the alignment tool to be secured relative to the loader within a range of relative rotational positions.

Another example may be found in a method of loading a stent onto a stent holder using an alignment tool. The method includes inserting the stent having a plurality of terminal end loops into the stent holder having a plurality of pins on which the terminal end loops are to be placed, and securing the alignment tool to the stent holder. The alignment tool includes a body, a plurality of securement arms extending from the body, each of the plurality of securement arms adapted to releasably secure the alignment tool relative to the loader, a plurality of alignment arms extending from the body, each of the plurality of alignment arms including a first end having an alignment slot and an opposing second end including a handle portion, the plurality of alignment arms movable between a closed configuration defining a minimum distance between the first end of each of the plurality of alignment arms and an open configuration defining a maximum distance between the first end of each of the plurality of alignment arms, and a biasing member that biases the plurality of alignment arms into the closed configuration. The method includes advancing the stent and moving one stent loop over each pin, compressing the stent onto the stent holder, and removing the alignment tool from the stent holder and the stent.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1A illustrates a stent loop positioned adjacent a pin on a stent holder before compression;

FIG. 1B illustrates the stent holder and stent of FIG. 1A with the stent loop correctly aligned over the pin and compressed;

FIG. 1C illustrates the stent holder and stent of FIG. 1A with the stent loop misaligned and compressed next to the pin;

FIG. 2 is a perspective view of a delivery system for delivering a replacement cardiac valve, including an illustrative alignment tool;

FIG. 3 is an enlarged view of a portion of the delivery system of FIG. 2;

FIG. 4 is a perspective view of the illustrative alignment tool shown in a closed configuration;

FIG. 5 is a perspective view of the illustrative alignment tool shown in an open configuration;

FIG. 6 is a perspective view of the illustrative alignment tool positioned relative to the alignment loops on the stent, with portions of the distal loader removed for clarity;

FIG. 7 is an enlarged view of a portion of FIG. 6, showing the illustrative alignment tool aligning an alignment pin with an alignment loop on the stent;

FIG. 8 is a perspective view of an illustrative alignment tool;

FIG. 9 is a perspective view of an illustrative alignment tool body; and

FIG. 10 is a perspective view of an illustrative alignment tool body.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Current artificial heart valves, such as the replacement valve and expandable anchor described in U.S. Pat. No. 8,992,608, must be loaded precisely into a delivery catheter, such as those described in U.S. Pat. Nos. 10,245,145 and 10,682,228, the disclosures of which are incorporated herein by reference. The artificial heart valve may include a stent portion with loops that must be compressed and aligned precisely in a delivery catheter just before implantation. The loading step can be complex and difficult and generally occurs in the Catheter Lab. The components, including pins on the stent holder and loops on the stent, are small and difficult to see to achieve precise alignment. The difficulties associated with the alignment steps increase the risk of misloading the valve with negative consequences for the loading time if the misload is identified and/or for the clinical result in terms of non-optimal implant positioning if the misload is not identified. Only one misload is generally permitted, and after a second misload the valve and delivery system must be discarded. The loading of the artificial heart valve and associated stent is a critical part of the implantation procedure, and improvements are desired.

Applicants have developed an automatic alignment tool that facilitates precise alignment of the stent portion of an artificial heart valve with the delivery system to enable smooth preparation and expedite the loading process. Automatic alignment of the valve with the delivery system will help the individual loading the valve, reducing stress and anxiety in a pressurized Catheter Lab environment. In some examples, the heart valve being loaded may be a transcatheter aortic valve replacement (TAVR) such as the ACURATE™ aortic valve system of Boston Scientific.

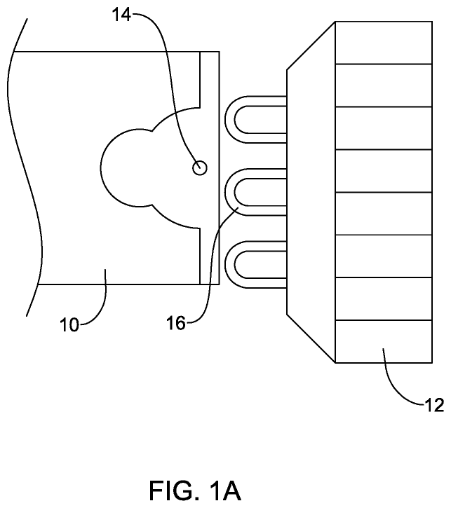

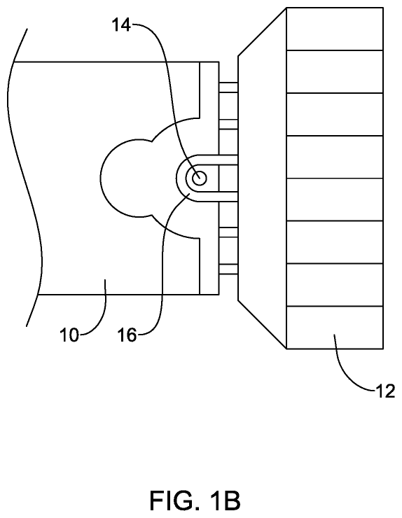

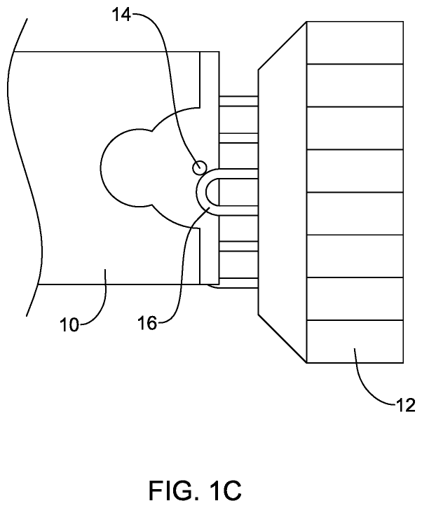

FIGS. 1A-1C illustrate loading the stent portion of a heart valve into a delivery device and some complications that may arise. FIGS. 1A-1C schematically show a portion of a stent holder 10 that is part of a delivery device, and a stent portion 12 of an artificial heart valve to be delivered using the delivery device. The stent holder 10 includes several pins 14 that need to be precisely aligned with corresponding loops 16 that are formed as a stent portion of the artificial heart valve 12. FIG. 1A shows a loop 16 that must be moved into alignment in order to engage the pin 14 prior to compression of the stent portion 12. The loops and pins are small and difficult to see. For example, the loops may be 1.5 mm and the pins may be 0.5 mm (0.060 inch and 0.020 inch, respectively), approximately the size of a ballpoint pen tip, which makes precise alignment difficult. FIG. 1B shows the correct alignment of the loop 16 over the pin 14 and compression of the stent. A complication that can lead to a missed loading of the valve is when one of the three valve loops 16 is mis-aligned with one of the three pins 14 on the stent holder 10, as shown in FIG. 1C. When the valve is then compressed, there is a potential for sheathing the valve on the stent holder 10 without all three loops being correctly engaged with the three pins. Once the device is sheathed, any misalignment may be difficult to see. If this device was deployed in a clinical scenario, the valve positioning and coaxial alignment could be compromised. The onus is on the individual loading the valve to identify any misalignments in a catheter lab and so alignment can be a significant cause of stress and anxiety. Even if the issue is identified the valve loading procedure must be restarted from the beginning leading to a scenario where the physician is waiting for the loaded valve and the TAVR procedure time is extended.

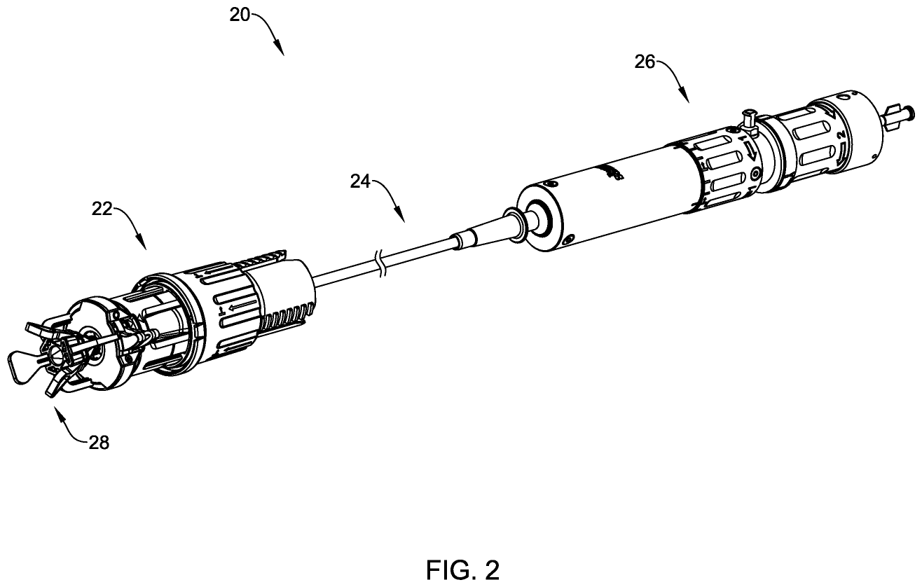

FIG. 2 is a perspective view of an illustrative valve delivery system 20 that may be used in preparing a replacement cardiac valve for delivery as well as for delivering and deploying the replacement cardiac valve. The illustrative valve delivery system 20 includes a distal loader tool 22 that may be used in preparing the replacement cardiac valve to be secured to the valve delivery system 20. As shown in FIG. 2, the replacement cardiac valve is disposed within the distal loader tool 22. The valve delivery system 20 includes a delivery catheter 24 and a handle assembly 26 that may be used in actuating the delivery catheter 24 when delivering the replacement cardiac valve. An illustrative alignment tool 28 is shown secured relative to the distal loader tool 22. The alignment tool 28 may be used in aligning (as shown in FIGS. 1A-1C) the pins 14 on the stent holder 10 (part of the delivery catheter 24) with the loops 16 that are formed within the stent portion 12 of the replacement cardiac valve prior to actuating the distal loader tool 22 to compress the replacement cardiac valve into position relative to the delivery catheter 24. The alignment tool 28 may be considered as providing hands-free operation when keyed to the distal loader 22.

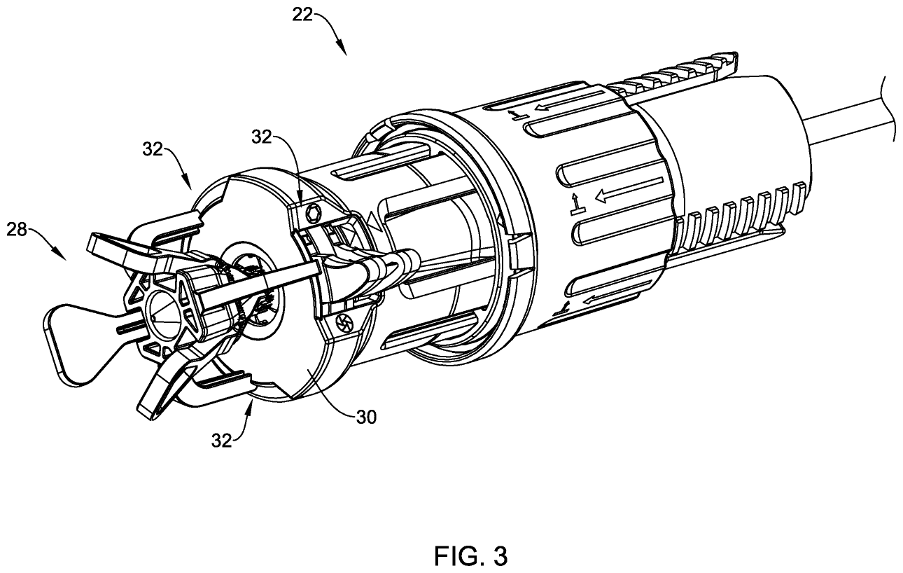

FIG. 3 is an enlarged view of the distal loader tool 22 and the alignment tool 28. The distal loader tool 22 includes a face place 30 that is located at a distal end of the distal loader tool 22. The face plate 30 includes several reduced diameter portions 32 that accommodate attachment of the alignment tool 28. As can be seen, each of the reduced diameter portions 32 extend partially around the circumference of the face plate 30, thereby allowing some adjustability in the rotational alignment of the alignment tool 28 with the face plate 30.

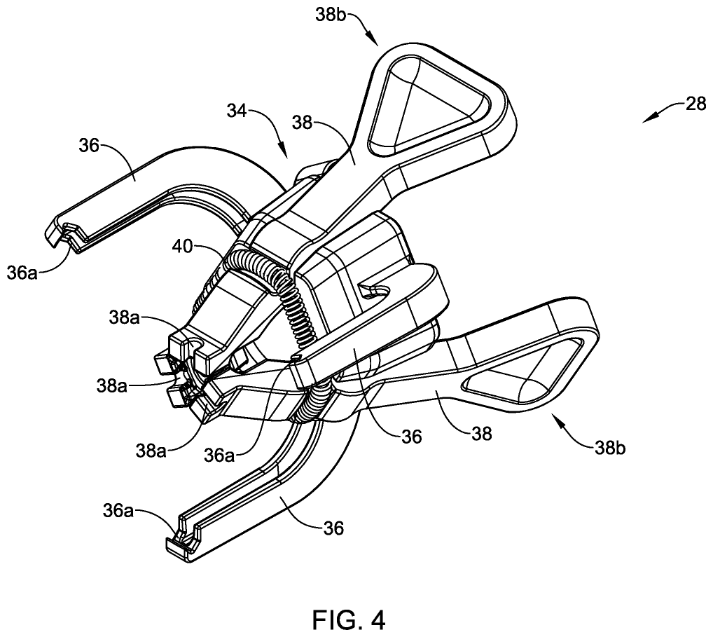

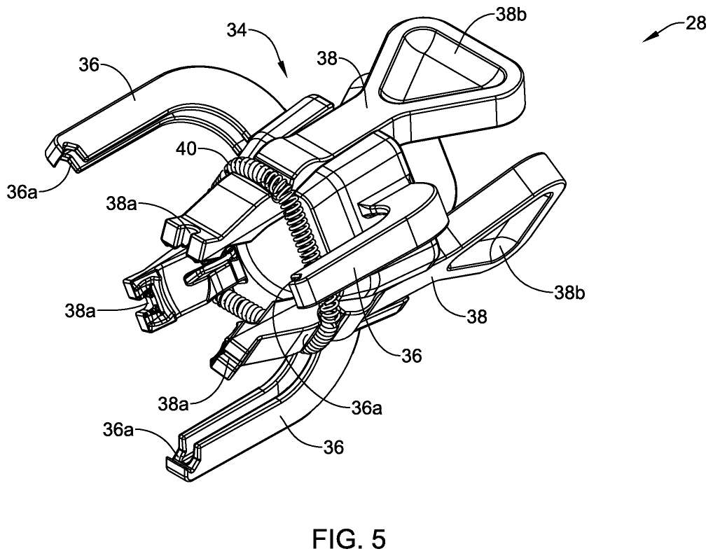

FIG. 4 is a perspective view of the alignment tool 28 in a closed configuration while FIG. 5 is a perspective view of the alignment tool 28 in an open configuration. The alignment tool 28 includes a body 34 and several securement arms 36 that extend outwardly from the body 34. In some cases, the securement arms 36 may be integrally or monolithically formed with the body 34. The body 34 and the securement arms 36 may be injection molded as a single structure, for example. In some cases, the securement arms 36 may be separately formed and subsequently secured to the body 34. While a total of three securement arms 36 are shown, this is merely illustrative, as in some cases the alignment tool 28 may have only one or two securement arms 36, or perhaps may have four or more securement arms 36. In some cases, however, the alignment tool 28 has three securement arms 36. Each of the securement arms 36 has an attachment point 36a by which the securement arm 36 may be releasably secured to the face plate 30 (of the distal loader tool 22). In some cases, the attachment point 36a of each of the securement arms 36 forms a frictional fit with the face plate 30. In some cases, the attachment point 36a of each of the securement arms 36 may be adapted to snap onto the face plate 30. The attachment point 36a of each of the securement arms 36 may be considered as providing sufficient holding power to hold the alignment tool 28 in position relative to the face plate 30 of the distal loading tool 22 while allowing the alignment tool 28 to be pulled off after the alignment tool 28 has accomplished its task.

The alignment tool 28 has several alignment arms 38 that are movably coupled to the body 34. In some cases, as can be seen in comparing FIG. 4 with FIG. 5, each of the alignment arms 38 are adapted to pivot relative to the body 34. In some cases, for example, the body 34 may include a pin (not shown) that fits into a corresponding slot formed in the alignment arm 38 that allows for each of the alignment arms 38 to pivot relative to the body 34, and thereby move from the closed configuration shown in FIG. 4 to the open configuration shown in FIG. 5. A biasing member 40 extends around each of the alignment arms 38 in order to bias each of the alignment arms 38 into the closed configuration.

In some cases, the biasing member 40 may be an annular spring or a garter spring. In some cases, the biasing member 40 may be an elastic member, such as a bungee or an O-ring. In some cases, rather than a single biasing member 40, the alignment tool 28 may have a separate biasing member coupled with each of the alignment arms 38. The biasing member 40 may be adapted to provide a particular biasing force to the alignment arms 38. As an example, the biasing member 40 may provide a biasing force in a range of 5 to 40 Newtons (N). The biasing member 40 may provide a biasing force in a range of 10 to 30 N. In an example, the biasing member 40 may provide a biasing force of about 22 N. In comparing FIG. 4 and FIG. 5, it can be seen that the biasing member 40 is stretched when moving from the closed configuration to the open configuration.

Each of the alignment arms 38 includes an alignment slot 38a at a first end of the alignment arm 38 and a handle portion 38b at an opposing second end. The handle portion 38b may be depressed in order to move each of the alignment arms 38 against the biasing force from the biasing member 40 and thus move from the closed configuration to the open configuration, for example.

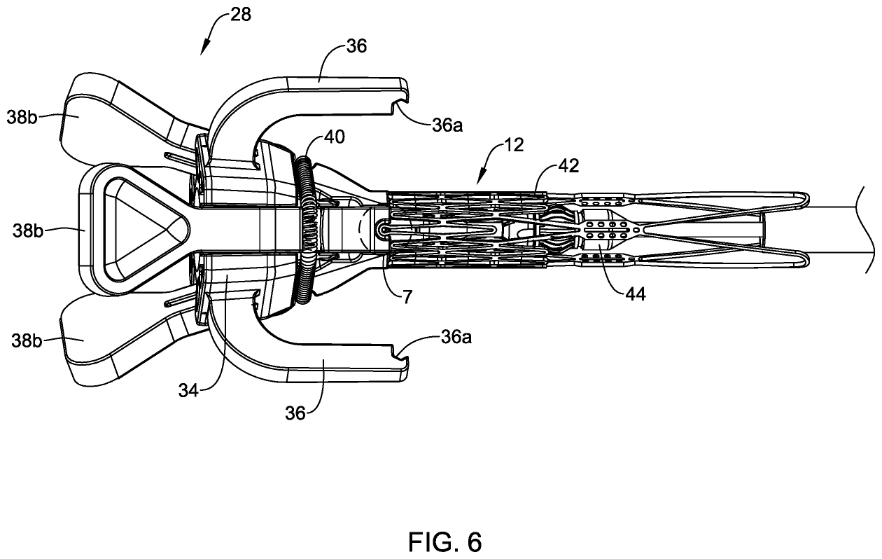

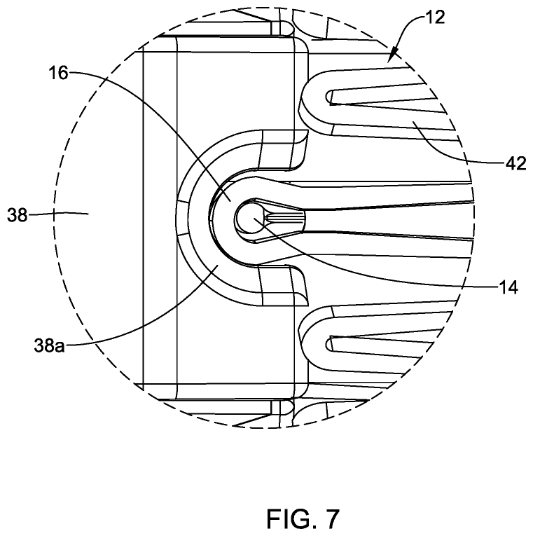

FIG. 6 is a perspective view of the illustrative alignment tool 28 positioned relative to the alignment loops 16 on the stent, with portions of the distal loader 22 removed for clarity and FIG. 7 is an enlarged view showing how the alignment slot 38a helps to align the loop 16 with the pin 14. In FIG. 6, a portion of a replacement cardiac valve 42 may be seen as disposed about a portion 44 of the delivery catheter 24 in the process of compressing the replacement cardiac valve 42, including the stent portion 12, with each loop 16 aligned with a corresponding pin 14. It can be seen that each loop 16 extends distally beyond the rest of the stent portion 12.

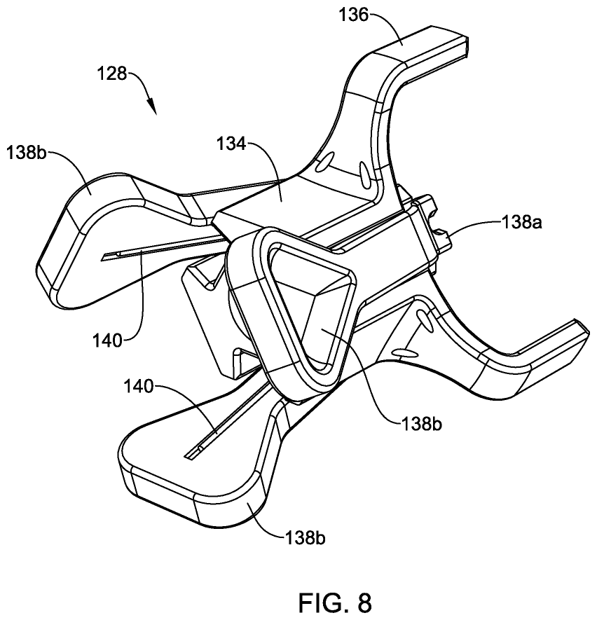

FIG. 8 is a perspective view of an illustrative alignment tool 128. Similar to the alignment tool 28, the alignment tool 128 includes a body 134, several securement arms 136 that are adapted for releasable securement to the face plate 30 of the distal loader 22, and several alignment arms 138 including an alignment slot 138a and a handle portion 138b. A unique feature of the alignment tool 128 relative to the alignment tool 28 is that the alignment tool 128 does not include a single biasing member 40, but rather includes a separate biasing member 140 that is disposed along each of the alignment arms 138. As an example, each of the biasing members 140 may be a torsion spring. Each of the biasing members 140 may be a straight element formed of a shape memory material that is biased to return to its straight configuration absent the application of any force attempting to deform the biasing member 140.

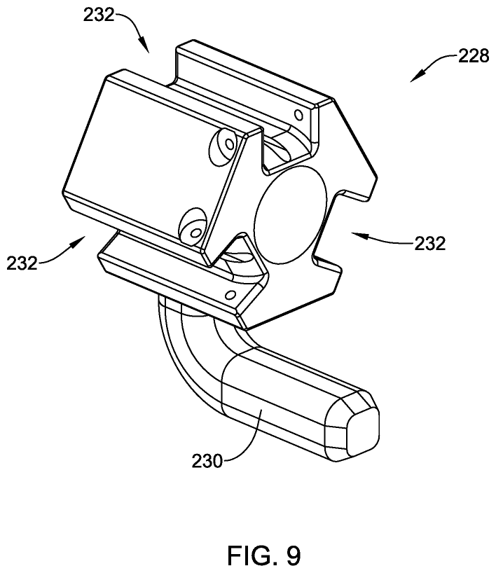

FIG. 9 is a perspective view of an illustrative alignment tool body 228 that may be used as part of an alignment tool. The alignment tool body 228 includes a securement arm 230 that may be adapted to releasably secure the alignment tool 228 to the face plate 30 of the distal loading tool 22. The securement arm 230 may align with a corresponding female boss extrusion on the loader faceplate. The alignment tool body 228 includes several slots 232 that are adapted to receive and accommodate alignment arms such as the alignment arms 38 or the alignment arms 138. The slots 232 are adapted to allow the alignment arms to pivot between an open configuration and a closed confirmation.

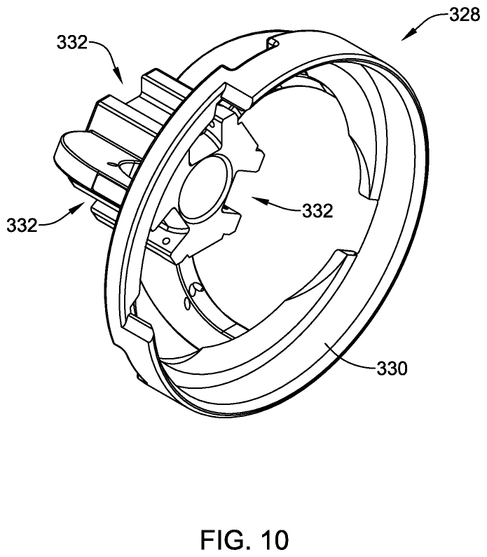

FIG. 10 is a perspective view of an illustrative alignment tool body 328. The alignment tool body 328 includes a securement ring 330 that may be adapted to releasably secure the alignment tool 228 to the face plate 30 of the distal loading tool 22. In some cases, the securement ring 330 permits rotation of the alignment tool relative to the distal loader in order to facilitate alignment. The alignment tool body 328 includes several slots 332 that are adapted to receive and accommodate alignment arms such as the alignment arms 38 or the alignment arms 138. The slots 332 are adapted to allow the alignment arms to pivot between an open configuration and a closed confirmation.

The alignment tool 28, 128 may be used to aid the user in aligning terminal stent loops on a stent or an artificial heart valve with pins on a stent holder. A method of using the alignment tool may include inserting the heart valve or stent having a plurality of terminal end loops into the stent holder having a plurality of pins on which the terminal end loops are to be placed. The alignment tool 28, 128 may be placed over the stent holder, as shown for example in FIG. 6. The stent may be moved into alignment with the stent loops aligned over the pins. The stent is then compressed onto the stent holder. The alignment tool 28, 128 may then be removed from the stent holder and the stent.

In some embodiments, one or more components of the alignment tool 28 (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as WIP35-N0 and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super-elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “super-elastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super-elastic plateau and/or flag region that may be seen with super-elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In some embodiments, one or more components of the alignment tool 28 (and variations, systems or components thereof disclosed herein), may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An alignment tool for loading a stent having a plurality of alignment loops onto a delivery catheter having a corresponding plurality of alignment pins adapted to be received within a corresponding alignment loop, the stent disposed within a loader having a loader housing, the alignment tool comprising:
a body;
a plurality of securement arms extending from the body, each of the plurality of securement arms adapted to releasably secure the alignment tool relative to the loader; and
a plurality of alignment arms extending from the body, each of the plurality of alignment arms including a first end having an alignment slot and an opposing second end including a handle portion, the plurality of alignment arms movable between a closed configuration defining a minimum distance between the first end of each of the plurality of alignment arms and an open configuration defining a maximum distance between the first end of each of the plurality of alignment arms;
wherein the plurality of alignment arms are biased into the closed configuration.

2. The alignment tool of claim 1, further comprising a biasing member that biases the plurality of alignment arms into the closed configuration.

3. The alignment tool of claim 2, wherein the biasing member comprises a garter spring extending around each of the plurality of alignment arms.

4. The alignment tool of claim 2, wherein the biasing member comprises an elastomeric member extending around each of the plurality of alignment arms.

5. The alignment tool of claim 2, wherein the biasing member comprises a separate biasing member secured relative to each of the plurality of alignment arms.

6. The alignment tool of claim 1, wherein the loader housing includes a face plate with which the plurality of securement arms are adapted to engage with when securing the alignment tool to the loader, the face plate adapted to permit the alignment tool to be secured relative to the loader within a range of relative rotational positions.

7. The alignment tool of claim 1, wherein the plurality of securement arms are monolithically formed as part of the body.

8. The alignment tool of claim 1, wherein the plurality of alignment arms are pivotably secured relative to the body.

9. An alignment tool for loading a replacement cardiac valve having a plurality of alignment loops onto a delivery catheter having a corresponding plurality of alignment pins adapted to be received within a corresponding alignment loop, the replacement cardiac valve disposed within a loader having a loader housing, the alignment tool comprising:
a body, the body adapted to be releasably securable to the loader;
a plurality of alignment arms extending from the body, each of the plurality of alignment arms including a first end having an alignment slot and an opposing second end including a handle portion, the plurality of alignment arms movable between a closed configuration defining a minimum distance between the first end of each of the plurality of alignment arms and an open configuration defining a maximum distance between the first end of each of the plurality of alignment arms; and
a biasing member adapted to bias the plurality of alignment arms into the closed configuration.

10. The alignment tool of claim 9, wherein the body includes one or more securement arms that are adapted to releasably secure the alignment tool relative to the loader.

11. The alignment tool of claim 9, wherein the body includes an annular structure that is adapted to releasably secure the alignment tool relative to the loader.

12. The alignment tool of claim 9, wherein the biasing member comprises an annular spring.

13. The alignment tool of claim 9, wherein the biasing member comprises an O-ring.

14. The alignment tool of claim 9, wherein the plurality of alignment arms are adapted to move from the closed configuration to the open configuration in response to an inward force applied to the second ends of each of the plurality of alignment arms.

15. The alignment tool of claim 9, wherein the second ends of each of the plurality of alignment arms are adapted to accommodate being squeezed together in order to move from the closed configuration to the open configuration.

16. The alignment tool of claim 9, wherein the plurality of alignment arms are adapted to pivot between the closed configuration and the open configuration.

17. The alignment tool of claim 9, further comprising a plurality of securement arms extending from the body, each of the plurality of securement arms adapted to releasably secure the alignment tool relative to the loader.

18. The alignment tool of claim 17, wherein the body and the plurality of securement arms are molded together.

19. The alignment tool of claim 17, wherein the plurality of securement arms are adapted to engage with a face plate of the loader when securing the alignment tool to the loader, the face plate adapted to permit the alignment tool to be secured relative to the loader within a range of relative rotational positions.

20. A method of loading a stent onto a stent holder using an alignment tool, the method comprising:
inserting the stent having a plurality of terminal end loops into the stent holder having a plurality of pins on which the terminal end loops are to be placed;
securing the alignment tool to the stent holder, the alignment tool having:
a body;
a plurality of securement arms extending from the body, each of the plurality of securement arms adapted to releasably secure the alignment tool relative to the loader;
a plurality of alignment arms extending from the body, each of the plurality of alignment arms including a first end having an alignment slot and an opposing second end including a handle portion, the plurality of alignment arms movable between a closed configuration defining a minimum distance between the first end of each of the plurality of alignment arms and an open configuration defining a maximum distance between the first end of each of the plurality of alignment arms; and
a biasing member that biases the plurality of alignment arms into the closed configuration;
advancing the stent and moving one stent loop over each pin;
compressing the stent onto the stent holder; and
removing the alignment tool from the stent holder and the stent.
